(19) United States
(12) Patent Application Publication
(10) Pub. No.: US 2009/0105688 A1
(43) Pub. Date: Apr. 23, 2009

(54) IMPLANTABLE ACCESS PORT WITH LUMINOUS GUIDE AND IDENTIFICATION SYSTEM

(76) Inventors: Jon T. McINTYRE, Newton, MA (US); Paul DiCARLO, Middleboro, MA (US)

Correspondence Address: FAY KAPLUN & MARCIN, LLP, 150 BROADWAY, SUITE 702, NEW YORK, NY, 10038, US

(21) Appl. No.: 12/209,377

(22) Filed: Sep. 12, 2008

Related U.S. Application Data
(60) Provisional application No. 60/973,569, filed on Sep. 19, 2007.

(51) Int. Cl. A61M 5/42

(52) U.S. Cl. 604/513; 604/116

(57) ABSTRACT

An implantable access port comprises a housing defining a fluid receiving chamber and a septum covering an opening of the housing to permit access to the fluid receiving chamber in combination with a light source coupled to the housing, the light source aimed so that, when the port is implanted subcutaneously in a desired orientation, light transmitted from the light source impinges on an area of skin adjacent to a location of the septum.

### PRIORITY CLAIM

This application claims the priority to the U.S. Provisional Application Ser. No. 60/973,569, entitled “Implantable Access Port with Luminous Guide and Identification System,” filed Sep. 19, 2007. The specification of the above-identified application is incorporated herewith by reference.

### BACKGROUND

bcutaneously implanted ports provide convenient fluid inlets to catheters while minimizing interference with mobility and reducing the probability of infections. However, it may at times be difficult to locate accurately the opening of such a subcutaneous port or to identify a type of the port.

### SUMMARY OF THE INVENTION

Described herein is an implantable access port comprising a housing defining a fluid receiving chamber and a septum covering an opening of the housing to permit access to the fluid receiving chamber in combination with a light source coupled to one of the housing and a catheter connected thereto, the light source aimed so that, when the port is implanted subcutaneously in a desired orientation, light transmitted from the light source impinges on an area of skin adjacent to a location of the septum.

In another aspect, a method for one of identifying and accessing a subcutaneously implanted port, comprises determining a general location of the port by palpation and engaging a power source of the subcutaneous port to activate a light source of the port in combination with locating a septum of the subcutaneous port guided by light from the light source.

### BRIEF DESCRIPTION OF THE DRAWINGS

FIG. 1 is a diagram showing an embodiment of an illuminated port according to the invention;

FIG. 2 is a diagram showing an embodiment of a power supply for illuminating a port according to the invention; and

FIG. 3 is a diagram showing a second embodiment of a power supply for illuminating a port according to the invention.

### DETAILED DESCRIPTION

The present invention may be further understood with reference to the following description and to the appended drawings, wherein like elements are referred to with the same reference numerals.

According to embodiments of the present invention, a subcutaneous port for fluid connection with a catheter or other medical conduit is provided with illumination that emanates therefrom enhancing accurate location of the septum and facilitating injection thereto. Such ports may be manufactured smaller as the illuminated guide feature enables more accurate location of the septum as compared to location by palpation, etc.

The illumination system according to the invention may also be used to facilitate identification of a type or purpose of a subcutaneous port. For example, a scheme may be devised whereby different patterns and/or colors of the illumination uniquely identify a manufacturer, type and model of port. Alternatively, a convention may be adopted by which ports having a specific illumination pattern and/or pattern are employed for specific purposes. For example, the illuminated identification feature of a port may indicate the specific anatomical structure into which the catheter attached thereto opens. This may be particularly useful for patients fitted with multiple ports, and/or for patients treated by personnel unfamiliar with their particular medical condition.

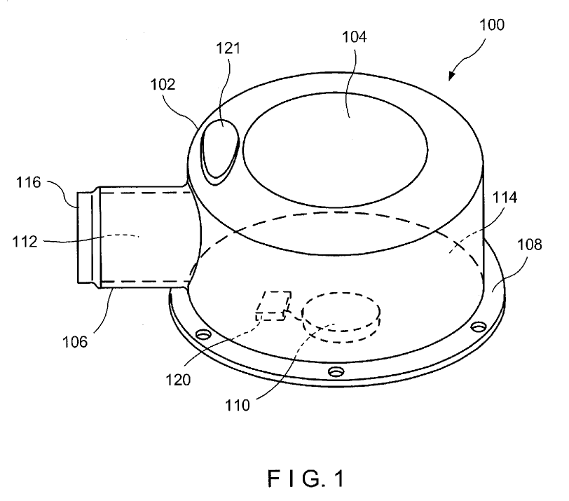

FIG. 1 shows an exemplary embodiment of a subcutaneous port with integral illumination according to the invention. The exemplary port 100 comprises a housing 102 defining a fluid chamber 114 into which therapeutic agents are injected and/or from which bodily fluids are withdrawn. As would be understood by those skilled in the art, the fluid chamber 114 is generally fluidly connected to a catheter or other conduit via a flow passage 112 which extends along an inlet/outlet 106. The fluid chamber 114 is open on one side, with the opening sealed by the septum 104 which, as would be understood by those skilled in the art is preferably formed of a material such as silicone or any other self-sealing material that permits repeated needle punctures without losing its ability to seal the opening to the chamber 114. The port 100 may further comprise a valve 116 located in the inlet/outlet 106, such as a PASV valve. A flange 108 may be used to suture the port 100 in place on an anatomical structure.

A light source 110 is disposed within the port 100 so that light emitted therefrom shines outward from the chamber 114, through the septum 104 toward a surface of the skin. That is, the light source 110 is oriented so that when the port 100 is implanted in a desired orientation with the septum 104 facing the skin, light from the light source 110 shines toward the skin illuminating the skin covering the septum 104. Thus, the position and orientation of the septum 104 may be accurately determined with the help of the illumination from light source 110 and the injection device may be inserted directly into the septum 104 via the illuminated portion of skin. The exemplary light source 110 is preferably selected to have an output power sufficient to remain visible after passing through the septum 104 and the skin. The port 100 further may further comprise a power source 120 to energize the light source 110, as described below. As would be understood by those skilled in the art, for embodiments in which light is to be transmitted through the port 100 or septum 104, one or both of the septum 104 and a top ring of the housing 102 surrounding the septum 104 are preferably formed of translucent elastomers.

In the exemplary embodiment the light source 110 is integral with the housing 102 and is disposed at the bottom of the fluid chamber 114 (i.e., a surface opposite an inner surface of the septum 104). Those of skill in the art will understand that one or multiple light sources may be placed at different locations in the port 100, to provide light passing through the septum 104 according to the invention. For example, the light source may be located anywhere inside the fluid chamber 114, or on top of the port housing 102, near the septum 104, etc. Multiple light sources may be disposed inside or around the outer surface of the septum 104.

The exemplary light source 110 may comprise one or more light emitting diodes (LED's) or other sources of light. For example, organic LED's, incandescent lights, chemically luminescent materials, or other light producing devices may be used according to the invention. LED's may be advantageous due to their low power consumption and long life. A light source producing little heating during operation is preferred, to avoid damaging surrounding tissue. Different embodiments of the light source according to the invention may employ luminescent gases, fluorescent or phosphorescent materials and other technologies to generate guide and identification light sufficiently strong to be visible transdermally.

The light source 110 may be turned on and off using conventional techniques. For example, a switch 121 may be placed on the port 100 to manually switch the light source 110. The switch 121 may be palpated through the skin and, after the general location of the device has been ascertained, pushed to energize the light source 110. A momentary switch may be used, so that the light source 110 de-energizes when the pressure on the switch 121 is removed. A latching switch may also be used, which will remain energized until the switch 121 is turned off by palpating it a second time. Furthermore, the light source 110 may be of a type that emits a pattern of light by intermittently turning on and off according to a predetermined sequence. As will be described below, external means for activating the light source 110 may also be used.

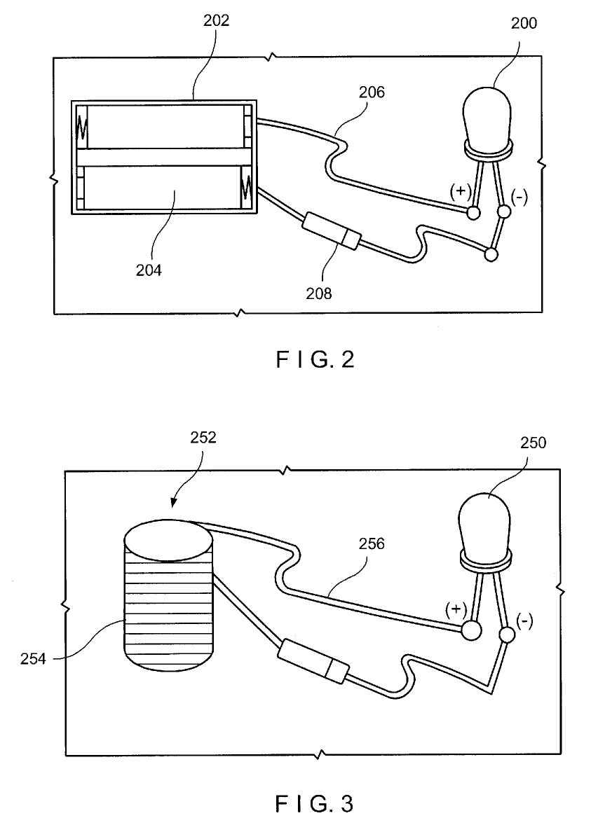

FIG. 2 shows, in one embodiment, light source 110 as including an LED 200 coupled to a power source 202 by conductors 206. The exemplary power source 202 comprises, for example, one or more batteries 204, which may be conventional or rechargeable. Those skilled in the art will understand that any other suitable energy producing device may be employed as the power source 202 coupled, for example, to a resistor 208 to limit the current drawn by the LED 200 or other light source.

An alternative activation mechanism for the light source 110 may comprise a coil disposed in the power source and an external RF emitting coil. As shown in FIG. 3, an LED 250 is connected by conductors 256 to a power source 252 comprising a coil 254. A portable external device (not shown) containing an RF emitting coil may be passed over the skin in the general location of the port 100 so that RF energy transmitted by the RF emitting coil induces a current in the nearby coil 254, energizing the LED 250 and illuminating the skin over the septum. Once the external device is removed from the vicinity of the port, the current stops and the LED 250 turns off.

The present invention has been described with reference to specific exemplary embodiments. Those skilled in the art will understand that changes may be made in details, particularly in matters of shape, size, material and arrangement of parts. Accordingly, various modifications and changes may be made to the embodiments. For example, the exemplary devices described may utilize alternative light emission and power generation means. The specifications and drawings are, therefore, to be regarded in an illustrative rather than a restrictive sense.

What is claimed is:

1. An implantable access port, comprising:
a housing defining a fluid receiving chamber;
a septum covering an opening of the housing to permit access to the fluid receiving chamber; and
a light source coupled to the housing, the light source aimed so that, when the port is implanted subcutaneously in a desired orientation, light transmitted from the light source impinges on an area of skin adjacent to a location of the septum.

2. The implantable port according to claim 1, further comprising a power source in the housing, the power source coupled to the light source.

3. The implantable port according to claim 1, wherein the light source includes at least one LED.

4. The implantable port according to claim 1, wherein the light source is disposed within the fluid chamber and wherein the septum is translucent, the light source being aimed to transmit light through the septum.

5. The implantable port according to claim 1, wherein the light source includes a plurality of LEDs.

6. The implantable port according to claim 5, wherein the plurality of LEDs is disposed around the septum.

7. The implantable port according to claim 1, further comprising a switch to selectively energize the light source.

8. The implantable port according to claim 7, wherein the switch is a palpable switch.

9. The implantable port according to claim 2, wherein the power supply comprises a battery.

10. The implantable port according to claim 2, wherein the power supply includes a coil electrically coupled to the light source, the coil being adapted to produce an induced current when an external RF emitting coil is placed in proximity to a portion of skin adjacent to the septum.

11. The implantable port according to claim 1, wherein the light source generates one of a pattern of light emissions and a color corresponding to one of a type of the port and an intended use for the port.

12. A method for one of identifying and accessing a subcutaneously implanted port, comprising:
determining a general location of the port by palpation;
engaging a power source of the subcutaneous port to activate a light source of the port; and
locating a septum of the subcutaneous port guided by light from the light source.

13. The method according to claim 12, wherein the power source is engaged by pressing a momentary switch.

14. The method according to claim 12, wherein the power source is engaged by pressing a latching switch.

15. The method according to claim 12, wherein the power source includes a coil and wherein the power source is engaged by placing an external RF emitting coil adjacent to a portion of skin over the septum to induce a current in the coil.

16. The method according to claim 14, further comprising disengaging the power source to de-activate the light source.

17. The method according to claim 16, wherein the power source is disengaged by pressing the latching switch a second time.

18. The method according to claim 12, further comprising analyzing one of a color and a pattern of illumination from the light source to determine one of a type and an intended use of the port.
